(19) United States
(12) Patent Application Publication
(10) Pub. No.: US 2023/0355119 A1
(43) Pub. Date: Nov. 9, 2023

(54) METHOD AND SOFTWARE TO GENERATE HEART RATE VARIABILITY POLAR MAP IMAGES WITH FILLED-IN PATIENT INFORMATION

(71) Applicant: Khalifa University of Science and Technology, Abu Dhabi (AE)

(72) Inventors: Mohanad A. R. ALKHODARI, Abu Dhabi (AE); Ahsan H. KHANDOKER, Abu Dhabi (AE); Leontios J. HADJILEONTIADIS, Abu Dhabi (AE); Herbert F. JELINEK, Abu Dhabi (AE)

(21) Appl. No.: 17/736,916

(22) Filed: May 4, 2022

(30) Foreign Application Priority Data
May 3, 2022 (GR) 20220100361

(51) Int. Cl. A61B 5/024; A61B 5/00; G16H 50/20

(52) U.S. Cl. CPC A61B 5/02405; A61B 5/742; A61B 5/7475; A61B 5/7275; G16H 50/20

(57) ABSTRACT

Techniques for determining heart rate variability (HRV) and subject data polar images are described. The techniques include determining data of a subject over a period of time that includes time segments and generating HRV feature values from the HRV data. Each one of the time segments is associated with at least one of the HRV feature values. The techniques also include generating, based on the HRV feature values and the time segments, a polar representation of the HRV data and outputting the polar representation as an image. The techniques further include inputting subject data (e.g., demographic data and/or clinical data of a subject) and using them to generate a filled-in color coded image. The generated image can be used further in a deep learning model to predict a heart failure category and decide on time segments or regions within the image that contribute toward the decision.

### CROSS-REFERENCE TO RELATED APPLICATIONS

This application claims priority to Greek Application No. 20220100361, filed May 3, 2022, the entire contents of which is hereby incorporated for all purposes in their entirety.

### BACKGROUND

Heart failure is a chronic pathological state that prevents the heart from pumping regularly to meet the body's need for oxygenated blood. It may be caused by the presence of coronary artery disease (CAD), which is characterized by an accumulation of plagues in the arteries feeding the heart leading them to become narrow or blocked. Globally, 64.3 million people are living with heart failure with an estimated 7.2 million deaths every year.

Heart failure patients suffer from a significant deterioration in the systolic function that may be evaluated based on the left ventricular ejection fraction (LVEF), which is the amount of blood pumped at each contraction of the left ventricle. Heart failure stages based on LVEF are variable and even though several guidelines have set certain thresholds to classify them, i.e., European Society of Cardiology (ESC), there are still no strict rules to decide due to the etiology of heart failure, treatment procedures, and overall clinical presentation of patients.

The most preferred tool for scanning LVEF-based heart failure is echocardiography. Although reliable, it requires expensive equipment, which decreases its availability in public healthcare sectors in less developed countries. Developing other indicators is thus an essential clinical aim. One such option is electrocardiography (ECG) and its corresponding heart rate variability (HRV) that is usually associated with the endocrine, autonomic nervous system (ANS), and intrinsic modulation of the cardiac electrophysiological rhythm. Due to the presence of CAD in heart failure patients, the autonomic regulator balance gets interrupted, and such behavior has been usually observed in literature through HRV analysis. However, deep understanding of the relation between HRV and heart rate failure is still not well defined. In addition, the conventional diagnostic procedures of heart failure are highly dependent on medical experts, which poses difficulties in the presence of big patient data in the form of images, signals, or clinical profiles.

The use of deep learning may be a promising approach to resolve the heavy dependency on medical expertise. Recent advances in deep learning have facilitated the growth of computerized algorithms in the diagnosis of heart failure. There have been many efforts to develop trained deep learning tools for the detection of heart failure in ECG signals. The use of ECG signals may be highly affected by the quantity of the recordings, and training models on long ECG signals may require high computational demands. Others have tried simplifying ECG signals into a corresponding short-term HRV data, that is a short sequence of consecutive R-peaks distances. Although short-term HRV is less complex, it does not include additional knowledge about cardiac variations throughout the circadian rhythm of the heart. Several works have also reported the use of deep learning in heart failure diagnostics using patient profiles. However, many demographical and clinical patient information could be highly overlapped among heart failure stages, more particularly when a narrower threshold is used to determine each stage.

### BRIEF SUMMARY

Embodiments of the present disclosure include a method of combining heart rate variability (HRV) data (which may be derived from electrocardiography (ECG) data) with subject data (e.g., demographic data and/or clinical data of a subject) into a single source of information. This information can be in the form of an image per HRV feature. Further, the information can be input to a deep learning model that, in response, generates a prediction of a heart failure category. This prediction can be mapped to particular contributing HRV feature values and subject feature values as an attention-based heatmap, thus, an understanding of the deep learning decision can be made, thereby aiding in heart failure diagnostics.

These illustrative examples are mentioned not to limit or define the disclosure, but to provide examples to aid understanding thereof. Additional embodiments and examples are discussed in the Detailed Description, and further description is provided there.

### BRIEF DESCRIPTION OF THE DRAWINGS

Features, embodiments, and advantages of the present disclosure are better understood when the following Detailed Description is read with reference to the accompanying drawings.

FIG. 1 is a schematic of a computer system for a heart failure analysis, according to one example of the present disclosure.

FIG. 2 illustrates an example of a flow for performing a heart failure analysis, according to one example of the present disclosure.

FIGS. 3A, 3B, and 3C are an illustration of generating an HRV polar image, according to one example of the present disclosure.

FIG. 4 is an illustration of a process of generating an image showing a polar representation of HRV features, according to one example of the present disclosure.

FIGS. 5A and 5B are an illustration of a process to generate an HRV feature polar image with filled-in subject data, according to one example of the present disclosure.

FIG. 6 is an illustration of a first panel of a user interface of an application, according to one example of the present disclosure.

FIG. 7 is an illustration of a second panel of the user interface, according to one example of the present disclosure.

FIG. 8 is an illustration of a third panel of the user interface, according to one example of the present disclosure.

FIG. 9 illustrates an example of a flow for generating an HRV polar image, according to one example of the present disclosure.

FIG. 10 illustrates an example of a flow for generating an HRV data and subject data polar image, according to one example of the present disclosure.

FIG. 11 illustrates an example of a flow for using a machine learning model to predict a heart failure, according to one example of the present disclosure.

FIG. 12 illustrates an example of a machine learning model usable to generate heart failure predictions, according to one example of the present disclosure.

FIG. 13 illustrates an example of a training a machine learning model to generate heart failure predictions, according to one example of the present disclosure.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced in other configurations, or without the specific details. Furthermore, well-known features may be omitted or simplified in order not to obscure the embodiment being described.

Embodiments of the present disclosure are directed to, among other things, combining heart rate variability (HRV) data with subject data (e.g., demographic data and/or clinical data of a subject) into a single source of information. This information can be used for generating a heart failure prediction for the subject. For instance, the approach may simplify HRV data and combine it with subject data using a polar representation per HRV feature. The polar representation can include time segments. Edges between time segments correspond to variations in the HRV feature over time. Areas within polar representations can indicate (e.g., via color coding) the subject data. An image showing the polar representation can be generated and presented at a user interface. The image (and, optionally, images of other HRV features similarly generated) can also be input to a machine learning model that, in response, outputs a heart failure prediction. The particular HRV values and subject data at particular times that contributed to the prediction can be determined and presented in the user interface.

Embodiments of the present disclosure provide several advantages. For instance, the embodiments provide an application for generating a combined representation of HRV feature values and subject feature values. Such a representation can be used for different purposes, including for heart failure diagnostics. In particular, the ECG/HRV data can used in a multi-dimensional (e.g., two-dimensional) manner instead of the conventional one-dimensional representation. The multi-dimensional polar representation allows for a better visual inspection of HRV variations in a selected proportion of time, thus, better evaluation of cardiac variations could be achieved. In addition, the proposed method integrates these variations with patient clinical information all in a multi-dimensional image instead of regular patient profiles stored in sheets as bulky data. The ability to create a color-coded clinical information simplifies the evaluation of patients with respect to their cardiac health condition, which would ensure better diagnosis when integrated altogether with the HRV variations all in one multi-dimensional polar image. When used with a machine learning model, a heart failure prediction can be generated with a high accuracy and the contributing factors can be identified. A single application can be implemented for receiving ECG and/or HRV data, presenting the polar representation of the HRV feature values, presenting an image that shows the combined HRV feature values and subject feature values, and presenting the heart failure prediction and the contributing factors.

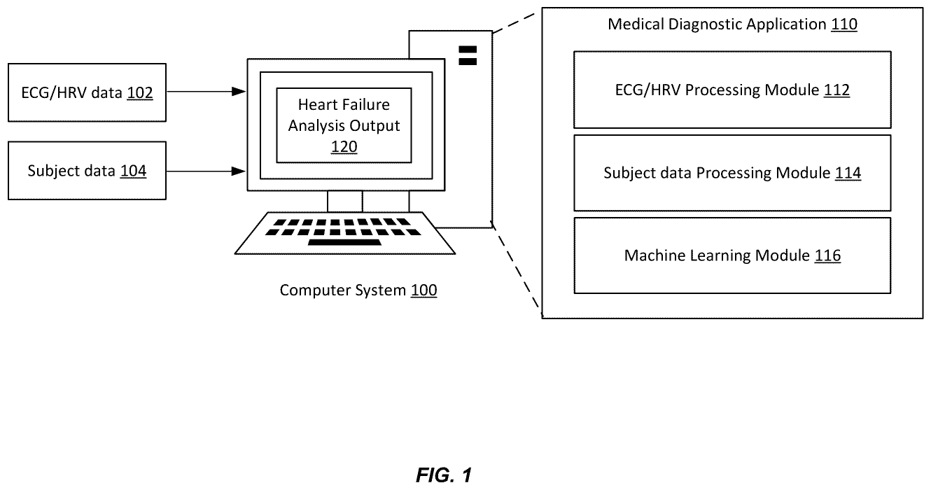

FIG. 1 illustrates an example of a computer system 100 for a heart failure analysis, according to one example of the present disclosure. In one example the computer system 100 can be a desktop computer system that includes a medical diagnostic application 110. With the computer system 100 is illustrated as a desktop computer, the computer system 100 may alternatively be a laptop, personal computer, tablet, or other device capable of presenting the user interface. Additionally, the computer system 100 may be a cloud-based system that includes a server (hardware or virtualized server) that executes one or more functionalities of the medical diagnostic application 110. In this case, the cloud-base system also provides an interface to a client device that is operable by a user and that presents a user interface to the medical diagnostic application 110. Yet in another example, the computer system 100 can be a distributed system, where some of the described functionalities (e.g., the processing of HRV data to generate a polar representation thereof) are executed on the client device, whereas other ones of the described functionalities (e.g., the machine learning processing) are executed on the server. Regardless of the implemented configuration of the computer system 100, this system 100 is capable of supporting various computing services including, for instance, information retrieval from information sources (e.g., diagnostics from clinicians stored in databases or remote storage devices), processing operations (e.g., to extract HRV features, generate representation of such features, etc.) and display operations (e.g., presentation of a user interface). In particular, the computer system 100 can include one or more processor(s), one or more memory storing computer-readable instructions, one or more network interfaces, one or more computer-readable storage media, and/or other computing components.

A user can input electrocardiography (ECG) data and/or HRV data 102 of a subject to the medical diagnostic application 110. The ECG/HRV data 102 represent measurements performed on the subject. Additionally, subject data 104 can be input to the medical diagnostic application 110. The subject data 104 represents demographic data and/or clinical data of the subject. Any or both types of data 102 and 104 can be input as a file storing such measurements or as link to a location where the file is stored. The medical diagnostic application 110 processes the ECG data and/or HRV data 102 and/or the subject data 104 using one or more modules, including an ECG/HRV processing module 112, a subject data processing module 114, and a machine learning module 116, to generate a heart failure analysis output 120. This output 120 can be presented at a user interface of the medical diagnostic application 110 and can include an image showing a polar representation of HRV feature values, an image showing a combination of a polar representation of HRV feature values and subject feature values, a heart failure prediction, and/or contributing factors to the heart failure prediction. Such types of the output 120 are further illustrated in the next figures.

In one example, the ECG/HRV processing module 112 can transform the ECG/HRV data 102 into a polar representation. If only ECG data is input, the ECG/HRV processing module 112 can extract HRV features (e.g., values over time for each HRV feature) therefrom and generate a polar representation for each HRV features based on the corresponding HRV feature values. If only HRV data is input, the ECG/HRV processing module 112 can directly extract the HRV features therefrom to generate the polar representation. Different HRV features are possible, as further described herein below. Of course, if both of ECG data and HRV data are input, the ECG/HRV processing module 112 can selectively extract HRV features from either or both types of data to generate the polar representation.

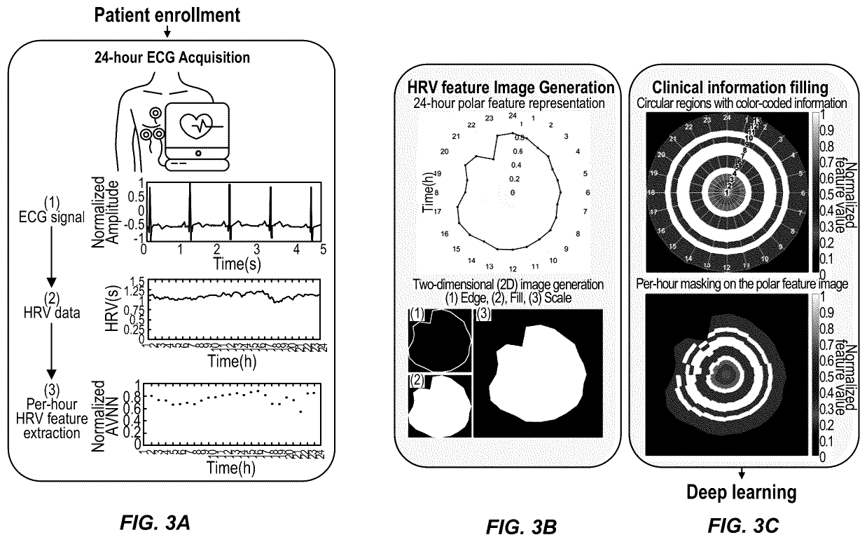

The polar representation can show the variations of HRV feature over a time period (e.g., 24 hours). In particular, the time period includes multiple time segments (e.g., 1-hour time segments). A value of the HRV feature is extracted for each time segment (e.g., every 1 hour is represented by an HRV feature value). The polar representation connects the start and the end of the time period by using a circle segmented according to the time segments (e.g., a circle including twenty-four segments). Each time segment is represented by a radial line extending from the center of the circle to the equivalent time segment value. For a time segment represented by a radial line and having a corresponding HRV feature value, the HRV feature is marked on the radial line depending on its value for the time segment. Two HRV feature values belonging to two adjacent time segments are connected by an edge, whereby this edge shows the variation of the HRV feature between the two time segments. An example of this polar representation using a time period of 24 hours, twenty-four 1-hour time segments, and an average normal-to-normal (AVNN) HRV feature is shown in FIG. 3B.

The subject data processing module 114 can process the subject data to determine subject features (e.g., demographic features and/or clinical features), generate values that represent these features (e.g., a value that represent an age and a value that represents gender) and generate a polar representation of the subject features based on the values. The polar representation here can similarly cover the same time period (e.g., 24 hours) segmented using the same time segments (e.g., 1-hour time segments). Color coding, pattern coding, numerical coding, and/or other visual presentations can be used to show the subject feature values. In some examples, the subject data processing module 114 may allow for selecting color palettes to compensate for color-blind users including protanopia, deuteranopia, and tritanopia. Generally, and unlike an HRV feature, the demographic features typically remain constant over time. Hence, their polar representation can result in edges between the time segments taking the form of concentric circles. An example of this polar representation using a time period of 24 hours, twenty-four 1-hour time segments, and thirteen subject features is shown in FIG. 3C.

The subject data processing module 114 can also receive a polar representation of an HRV feature as an input to then generate a combined polar representation of both the HRV feature values and the subject feature values. Alternatively, the polar representation of the subject features is output to the ECG/HRV processing module 112 that then performs the combination. In another illustration, the functionalities of the ECG/HRV processing module 112 and the subject data processing module 114 can be combined in a single module. Regardless of how the functionalities are implemented, an image of the polar representation of the HRV feature values, an image of the polar representation of the subject feature values, and/or an image of the combined representation of both the HRV feature values and the subject feature values can be generated. Any or a combination of these images can be further processed by the machine learning module 116. Additionally or alternatively, polar representations and/or the raw HRV feature values and raw subject feature values (independently of their polar representations) can be input to the machine learning module 116. Further, multiple images and/or polar representations corresponding to multiple HRV features can be included in the input.

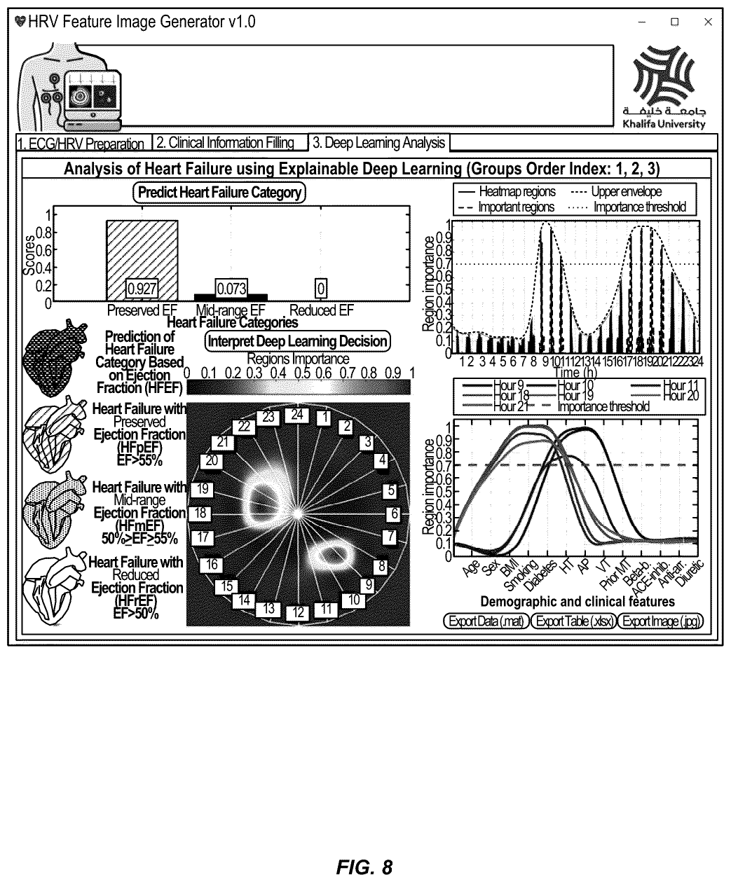

The machine learning module 116 can include an instance of a machine learning model (e.g., a convolutional neural network) trained to generate heart failure predictions. For example, the machine learning model can be a classifier that outputs, for the subject and based on the received input (e.g., any of the images, the polar representations, and/or raw features specific to the subject), a heart failure prediction across heart failure categories (e.g., ejection fraction (HFEF), preserved ejection fraction (HFpEF), mid-range ejection fraction (HFmEF), reduced ejection fraction (HFrEF)). The prediction can include a likelihood per heart failure category. Further, machine learning module 116 can indicate the contributing features to the prediction of the machine learning model in a form of a heatmap. The heatmap can mimic the human perception in analyzing objects as it returns most important regions that derived the decisions made by the trained model. Such prediction and heatmap are further illustrated in FIG. 8.

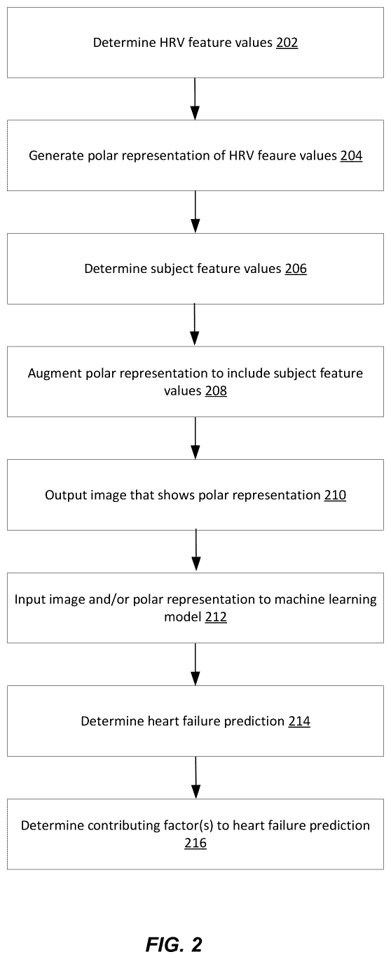

FIG. 2 illustrates an example of a flow for performing a heart failure analysis, according to one example of the present disclosure. Operations of the flow chart can be performed by a computer system, such as the computer system 100. The instructions for performing the operations can be stored as computer-readable instruction on a computer-readable medium of the computer system. The one or more memory storing computer-readable instructions that, upon execution by the one or more processors, configures the computer system to perform the operations of the flow. As implemented, the instructions represent modules that include code executable by processor(s) of the computer system. While the operations are illustrated in a particular order, it should be understood that no particular order is necessary that one or more operations may be reordered.

In an example, the flow includes operation 202, where the computer system determines values of an HRV feature of a subject. For instance, ECG data and/or HRV data are received by a medical diagnostic application based on a user input at a user interface thereof. As needed, the medical diagnostic application processes the ECG data to generate HRV data and extracts HRV features by applying a statistical measure to the HRV data based on time segments such that a value for each HRV feature is determined per time segment. The HRV feature can be a time domain or a frequency domain feature.

In an example, the flow includes operation 204, where the computer system generates a polar representation of the values of HRV feature. For example, the HRV feature values can be mapped to locations on radial lines, where each radial line represents a time segment. Edges can connect adjacent locations to form a boundary of the HRV feature values.

In an example, the flow includes operation 206, where the computer system determines subject feature values. For example, subject data that include demographic data and/or clinical data of the subject are received by the medical diagnostic application based on a user input at a user interface thereof. Subject features can be extracted therefrom and can include, for instance, age, gender, body mass index, smoking, diabetes, hypertension, angina pectoris, ventricular tachycardia, prior myocardial infarction, beta-blockers, ACE-inhibitors, anti-arrhythmics, and diuretics. The original value for each of these features is determined and can be mapped to an updated value according to a coding rule. The updates values represent form the subject feature values.

In an example, the flow includes operation 208, where the computer system augments the polar representations of the HRV feature values to include the subject feature values. For instance, a polar representation of the subject demographics can be generated using the same time segments, one or both of the polar representations can be scaled, and the two polar representations are combined. In addition, image masking techniques can be used as further described in the next figures.

In an example, the flow includes operation 210, where the computer system outputs an image that shows the polar representation. An image can be output for each HRF feature. For an HRV feature, its corresponding image can include a visual representation of values of the HRV feature and the values of the subject features according to their combined polar representation. For instance, the image shows a shape having outer boundary that follows the edges that connect the HRV feature values. Areas within the shape are segmented according to the time segments and portions of these areas are color coded (or some other visual presentations are used) to show the subject feature values. This image can be presented at a user interface of the medical diagnostic application. The image color coding can be changed from a regular mode to compensate for color-blind users including protanopia, deuteranopia, and tritanopia.

In an example, the flow includes operation 212, where the computer system inputs the image and/or the polar representation to a machine learning model. For instance, the machine learning model can be a deep learning model trained to generate heart failure predictions.

In an example, the flow includes operation 214, where the computer system determines a heart failure prediction for the subject. For instance, the output of the machine learning model includes the heart failure prediction. This output can be presented at a user interface of the medical diagnostic application.

In an example, the flow includes operation 216, where the computer system may determine contributing factor(s) to the heart failure prediction. For instance, the contributing factor(s) can be extracted from a layer of the machine learning model (e.g., a max pooling layer) and presented as a heatmap on a user interface of the medical diagnostic application.

Accordingly, embodiments of the present disclosure represent a novel technique to combine ECG, HRV, and patient profiles as a single source of information in the form of an image. The technique generates HRV polar map images with filled-in patient demographical and clinical information. This would reduce the pressure on medical experts to read through multiple sources of medical data (ECG, HRV, and patient information) by combining them altogether in a single image. It is made simple for the use by medical doctors, patients, or researchers by integrating it within a user-friendly software (e.g., in .exe format).

To illustrate, a user inputs either an ECG signal or extracted HRV data to an application that embodies the technique. The application the extracts pre-defined segments from the HRV data, e.g., per-hour, to obtain corresponding HRV features and plot them in a polar plot representation. The polar representation shows time variations in the selected feature's amplitude. The application also converts the polar plot into a two-dimensional (2D) binary image. In addition, the application requests the user to input patient demographical and clinical data. Patient profiles can be filled in within the generated HRV polar image through a color-coding mechanism, such as the one shown in Table 1 below. The final image is a 2D representation of the variations of the HRV feature across the selected segmentation scenario (in this case the 24-hour cardiac cycle) with filled-in and color-coded patient profile. The application also allows for different selections of color palettes to suit color-blind users with protanopia, deuteranopia, and tritanopia. The generated image is a polar representation of the patient's HRV data with edges corresponding to per-hour feature variations and interior of filled-in color-coded demographical and clinical information. Instead of relying only on one-dimensional (1D) HRV data, the application transforms the data into a 2D image with pre-defined segments that allows the inclusion of longer recordings in a single image. In addition, the application includes patient profiles as part of this image to ensure a complete diagnosis of patients' heart condition relative to their clinical status.

TABLE 1
Color-coding mechanism for patient demographical and clinical
information within the generated polar image.

| Circle | Demographic/ | | Additional normalization |
|---|---|---|---|
| label | clinical information | Color codes | notes |
| 1 | Age, yrs | Blue: <35 | Patient's age is divided by |
| | | Purple: 35-65 | 100 to normalize as [0-1] |
| | | Orange: 65-100 | Patients older than 100 are |
| | | White: >100 | fixed to 1 |
| 2 | Sex | Blue: Male | Male patients are set to 0.3 |
| | | Pink: Female | Female patients are set to 0.6 |
| 3 | Body Mass Index, | Blue: <25 | Normalize using a sigmoid |
| | kg/m² | Purple: 25-30 | function with: |
| | | Orange: 30-35 | Mean = 27.28 |
| | | White: >35 | Standard deviation = 3.45 |
| 4 | Smoking | Blue: No | No is set to 0.2 |
| 5 | Diabetes | White: Yes | Yes is set to 1 |
| 6 | Hypertension | | |
| 7 | Angina Pectoris | | |
| 8 | Ventricular | | |
| | Tachycardia | | |
| 9 | Prior Myocardial | | |
| | Infarction | | |
| 10 | Beta-blockers | | |
| 11 | ACE-inhibitors | | |
| 12 | Anti-arrhythmics | | |
| 13 | Diuretics | | |

Furthermore, the application can provide the users with an additional ability to test for their heart failure status, if any. The users can input the generated images to a pre-trained deep learning model to predict heart failure category, i.e., heart failure with preserved ejection fraction (HFpEF), heart failure with mid-range ejection fraction (HFmEF), and heart failure with reduced ejection fraction (HFrEF). The 2D images are advantageously used instead of 1D data to transform the regular black-box prediction mechanism into explainable deep learning capable of deriving knowledge on deep learning decisions with respect to heart failure classifications. The whole process is integrated within an easy-to-use user interface of the application to better translate the computational algorithms into clinical cardiology applications.

As such the embodiments of the present disclosure combine ECG, HRV, and patient profiles as a single source of information (image). This would reduce the pressure on medical experts to read through multiple sources of medical data (ECG, HRV, and patient information) by combining them altogether in a single image. The embodiments also transform 1D ECG/HRV signals into interactive 2D color images. The embodiments allow filling patient demographical and clinical profiles in the generated 2D images through a color-coding mechanism. The integration of HRV timely changes and patient profiles can be used in a new protocol for the clinical diagnosis of heart diseases. Further, the embodiments provide the capability of generating a single 2D image for long or short recordings (with proper segmentation), thus, reducing the complexity and bulkiness in analyzing cardiac conditions. Explainable deep learning model that reads input 2D HRV images to predict LVEF-based heart failure categories are supported, thus, providing an insight on heart failure condition from a trained machine perspective. The different algorithms and methods of the embodiments can be wrapped up in a user-friendly application (e.g., in .exe format of size 22.5 MB only). The application allows establishing a connection between complicated computations/algorithms and medical doctors or non-technical users. The application need not necessitate any manual interference except for specifying the inputs (ECG/HRV signals and patient profile) and selecting tasks. The application provides the users with the ability to store the analysis of every step (e.g., in .xlsx) for medical doctors as patient information, for visual inspection (e.g., in .png format), and for researchers and future works (e.g., in a .mat format). The application also supports deep learning capabilities to analyze heart failure if needed by the user and to provide an explainable mechanism of the trained model decisions.

The embodiments enable the transformation of the ECG/HRV signals into much simpler representations to ease the visual interpretation of cardiac function variations. HRV features can be plotted in a polar map representation, the plot converted into a 2D image, and the generated image filled with color-coded patient profiles. The integration of three patient information, including HRV feature values, timely changes, and demographical/clinical information, within a single 2D color image can support a new protocol for the clinical diagnosis of heart diseases. Instead of relying on quantitative ECG signals or HRV values, which could be bulky if analyzed on long-term recordings, i.e., 24 hours, the embodiments provide a simpler single image visualization capable of providing all needed information about the patient to the doctor.

In an example, to convert patient information into more visual-friendly representations, color coding rules were applied for each demographical and clinical information (e.g., per Table 1). The information was normalized according to these rules in a way that forms unique color codes for each variable. For age (in years), the value was divided by 100 to get a value between 0 and 1. If the patient's age was more than 100, their age was set to 1. Accordingly, the color representations for age can be described as: blue <35, purple 35-65, orange 65-100, and white >100. For sex, male patients were coded as 0.3 (blue) and female patients as 0.6 (pink) to be easily discriminated through visual inspection. For BMI (in kg/m2), all values were normalized using a regular sigmoid function with the mean (27.28) and standard deviation (3.45) of the current dataset. Thus, BMI was represented in colors as: blue <25, purple 25-30, orange 30-35, and white >35. For categorical variables (yes/no answers), including smoking, diabetes, hypertension, angina pectoris, ventricular tachycardia, prior myocardial infarction, beta-blockers, ACE-inhibitors, anti-arrhythmics, and diuretics, a value of 0.2 was given to the no answer (blue) and a value of 1 was given to the yes answer (white).

HRV features were extracted HRV features on hourly basis from time domain: average normal-to-normal (NN) interval (AVNN, ms), standard deviation of the NN intervals (SDNN, ms), square root of the mean of the sum of squares of differences between adjacent NN intervals (RMSSD, ms), percentage of NN intervals more than 50 ms (pNN50, %), and standard error of the average NN interval (SEM, ms), frequency-domain: slope of the linear interpolation of the spectrum for frequencies less than the very-low frequency (VLF) band upper bound (BETA), high frequency (HF) normalized power (HF Norm, %), peak frequency of the HF band (HF Peak, Hz), power in the HF band (HF Power, ms²), low frequency (LF) normalized power (LF Norm, %), peak frequency of the LF band (LF Peak, Hz), power in the LF band (LF Power, ms²), ratio between the LF power and the HF power (LF/HF), total power in both frequency bands (Total Power, ms²), VLF normalized power (VLF Norm, %), and power in the VLF band (VLF Power, ms²), non-linear metrics: standard deviation of the NN intervals along the perpendicular to the line-of-identity (SD1, ms), standard deviation of the NN intervals along the line-of-identity (SD2, ms), de-trended fluctuations analysis for the low-scale slope (alpha1), de-trended fluctuations analysis for the high-scale slope (alpha2), and complexity of physiological time-series signals (Sample Entropy), and fragmentation metrics: percentage of inflection points in the NN intervals (PIP, %), acceleration and deceleration segments inverse average length (IALS), percentage of short segments (PSS, %), and percentage of alternation segments (PAS, %).

The generation of the HRV feature image includes two components: the transformation of HRV features values to a 2D polar image and the filling-in with color coded patient information. Initially, each HRV feature value was normalized and drawn as a polar map representing a 24-hour clock. Each per-hour feature value was converted from polar to Cartesian (x, y) coordinates. Then, the Cartesian points were mapped to a 2D space with dimensions of 512×512 and connected to form the outline of the feature image. After filling the outline with a binary value of 1 (background with 0), the generated image is re-scaled to its original dimensions relative to the polar map plot. The re-scaling factor (a value between 0 and 1) was calculated by measuring the difference between every point on the Cartesian coordinates and a circular base plot that represents the maximum possible value of every feature after normalization, i.e., 1 in a scale from 0 to 1.

To fill-in color coded patient information, thirteen circular rings are generated (corresponding to thirteen types of patient information) that extend from the center to the edges of the image. Each circular ring was filled with a color coded demographical or clinical variable with a pre-defined order. For each hourly pie-shaped segment, the binary HRV feature segment and the circular rings segment were extracted. To be able of masking the hourly circular rings segment on the binary HRV feature segment, scaling was used to make them the exact same size. To generate the final filled-in HRV feature image, all hourly scaled segments are combined, and their edges smoothed to form a unique connected shape without any sharp edges.

FIGS. 3A, 3B, and 3C are an illustration of generating an HRV polar image, according to one example of the present disclosure. The process may be performed by an application (e.g., the medical diagnostic application 110). The process may start with a patient enrollment (e.g., a subject for which heart failure diagnostic is desired). The application may generate HRV map images with filled-in subject data. In particular, the application may receive an ECG signal or extracted HRV data, as shown in FIG. 3A. The application may then extract pre-defined segments from the HRV data. In one example, the predefined segments may be per-hour segments. The HRV segments may be obtained and correspond to HRV features segmented along the per-hour segments. The application may convert the HRV segments into a polar representation. The polar representation may show the time variations in the selected HRV feature's amplitude, as shown in the top part of FIG. 3B. In one example, the application may convert the polar representation into a two-dimensional image, as shown in the bottom part of FIG. 3B. Further, the application may generate a polar representation of subject features, as shown in the top part of FIG. 3C. These features may be converted into per-hour segments and masked to the polar representation of the HRV features to generate the HRV polar image with the filled-in subject data, as shown in the bottom part of FIG. 3C. The process may end by outputting this image to a deep learning model.

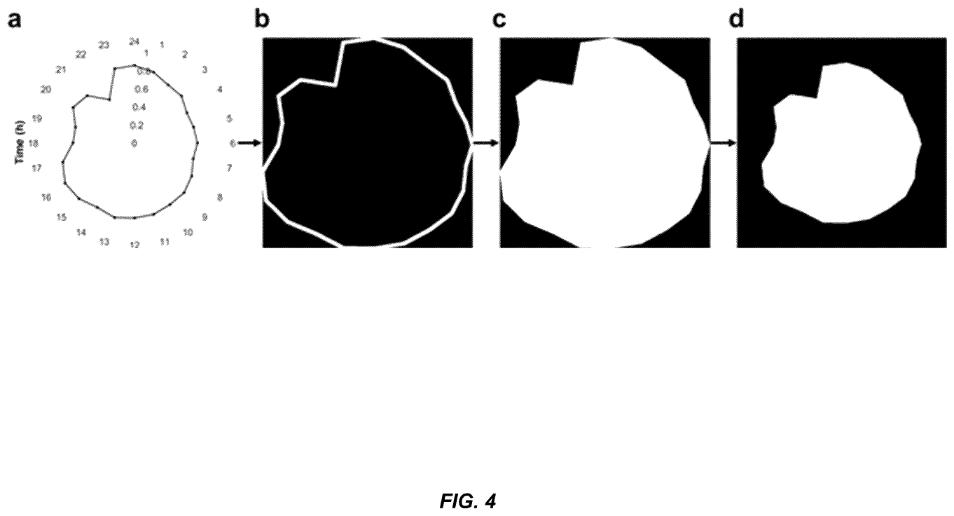

FIG. 4 is an illustration of a process of generating an image showing a polar representation of HRV features, according to one example of the present disclosure. The process may be performed by an application (e.g., the medical diagnostic application 110). The application can generate, from HRV data, a polar representation of value of an HRV feature segmented according to time segments within a time period (e.g., one-hour time segments within 24 hours), as shown in part (a) of FIG. 4. In one example, an HRV feature for a time segment (e.g., the second hour) can be the average peak-to-peak of HRV data that corresponds to the time segment (e.g., the HRV data measured and/or extracted for the second hour). In the polar representation, adjacent HRV feature values can be connected using edges. The application then converts the polar representation into a two-dimensional edge image as shown in part (b) of FIG. 4, whereby this image shows a boundary that traces the edges. Other elements of the polar representation may not be shown in the two-dimensional edge image. The application further converts the two-dimensional edge image representation into a two-dimensional filled image as shown in part (c) of FIG. 4, whereby, in this image, the application fills in the area defined by the boundary with a particular color. For instance, the filled image is a binary image, where the area has one color and the remaining portion of the image has another color. The application scales the two-dimensional filled image representation into a two-dimensional scaled image as shown in part (d) of FIG. 4, whereby the scaling using maximum possible values (e.g., a circle with a diameter of one).

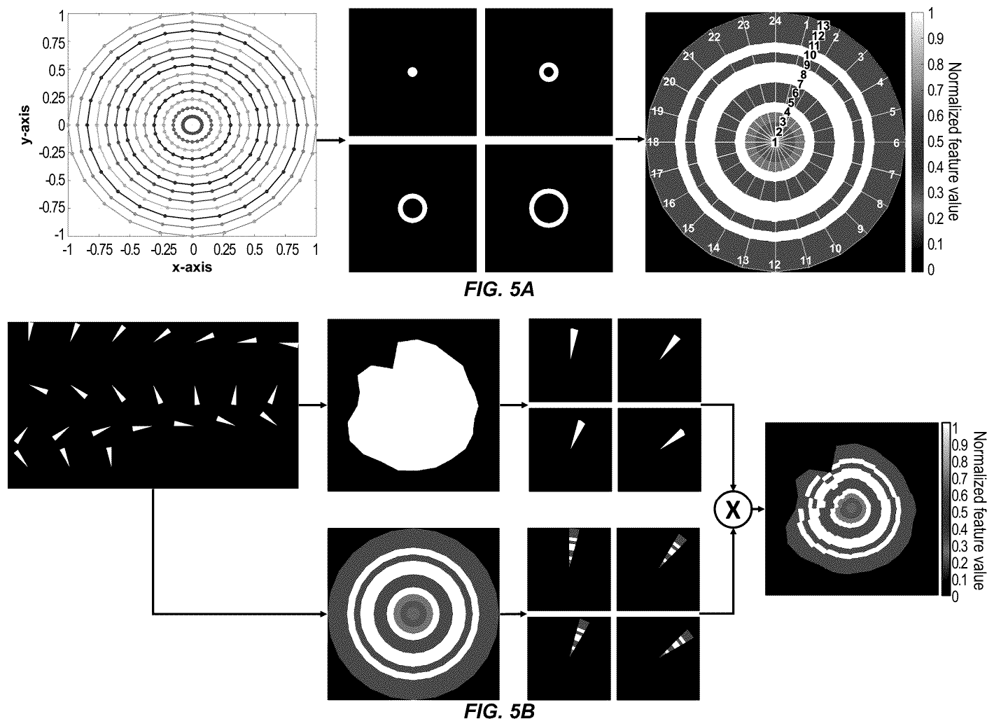

FIGS. 5A and 5B are an illustration of a process to generate the HRV feature polar image with filled-in subject data, according to one example of the present disclosure. The process may be performed by an application (e.g., the medical diagnostic application 110). In one example, the application determines subject features, including demographic features and clinical features of a subject. The application can convert the values of such features into rings to represent a full circle having maximum possible feature value (e.g., one), as shown in FIG. 5A. The application arranges a 24-hour triangular shapes to represent each hourly segment, as shown in FIG. 5B. Each hourly segment is masked on the HRV polar image to extract the corresponding shape for the selected hour. Similarly, the application masks each hourly segment on the full circle of the color-coded features to obtain hourly segments of clinical information. The application then rescales the color-coded masks to match with the HRV masks and multiplies them by each other to obtain the color-coded HRV mask. The addition of all masks generates the HRV feature image with filled-in subject data.

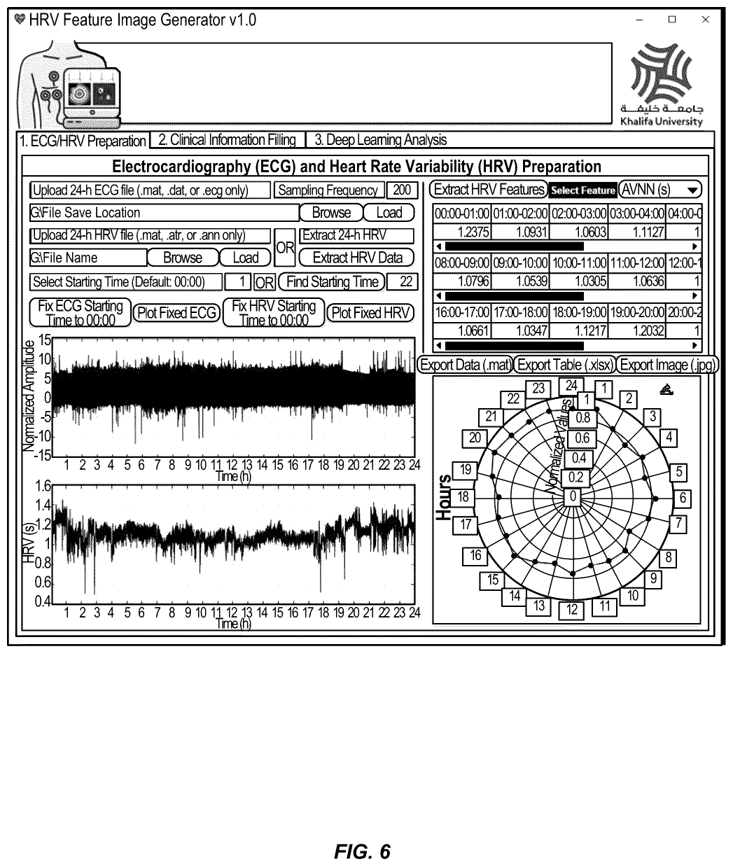

FIG. 6 is an illustration of a first panel of a user interface of an application that performs the above processes (e.g., the medical the medical diagnostic application 110), according to one example of the present disclosure. The user interface can be a graphical user interface (GUIs) that presents different panels, each corresponding to a particular set of user functionalities. The panels can be organized using a tab-based format, although other types of organizing the panels (e.g., side-by-side windows) are possible. In the illustration of FIG. 6, the first user interface presents the first panel for inputting and showing ECG and HRV data, extracted HRV features, and a polar representation of each HRV feature. In one example, the ECG/HRV data may be input to the application as .mat, .dat, .ecg, .atr and/or .ann file formats. The first panel can also include an option to export the ECG/HRV data, the HRV features, and/or an image version of the polar representations of the HRV features. The first panel allows the user to request an automatic extraction of HRV data from the loaded ECG signal. In addition, the first panel allows the user to fix the recording to start at 12:00 AM either manually or with a built-in algorithm based on Cosinor fitting. Upon the application extracting HRV features and plotting the corresponding polar representations, a second panel of the user interface becomes operable, where the user can input clinical information of the patient and generate the filled-in HRV feature images. This second panel provides the user with the ability to select their preferred color palette. The color palettes are Regular (similar to the colormap used for PET imaging), color-blind: protanopia, color-blind: deuteranopia, and color-blind: tritanopia.

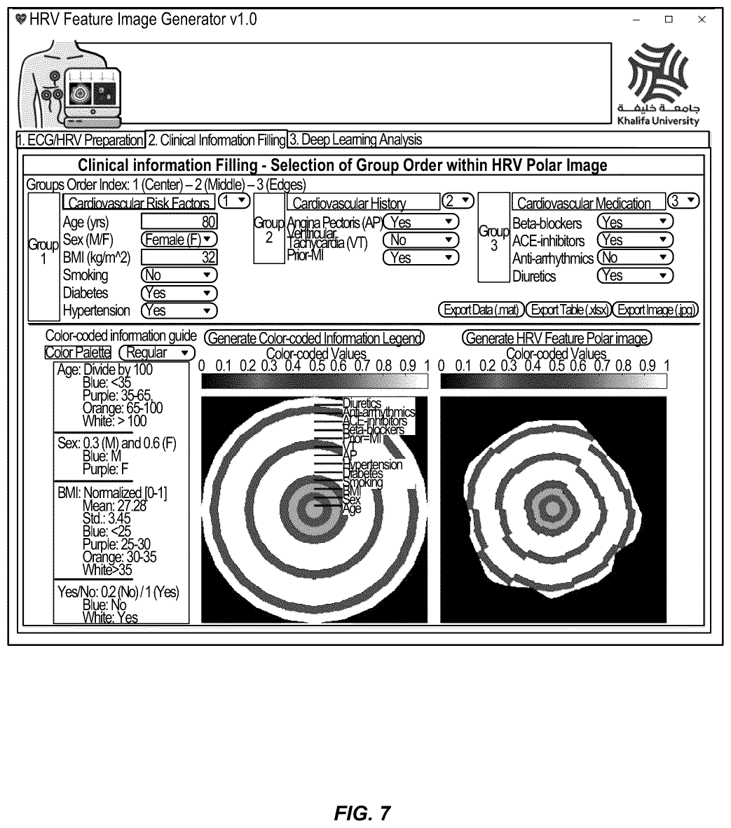

FIG. 7 is an illustration of the second panel of the user interface, according to one example of the present disclosure. In one example, the second panel can be used for inputting subject data, presenting subject features, presenting a polar representation of the subject features, and presenting, for each HRV feature, a polar image of the HRV feature values with filled-in subject features. The subject data input can follow the format shown in Table 1 above. An example of such an input is shown in Table 2 below. The application can default to using circle labeling/order, however, the second panel can provide the user with an option to change this in any order of preference. In one example, the second panel allows changing the visual representation of the subject features in color modes such as color-blind modes comprising protanopia, deuteranopia, and/or tritanopia. The second panel can also include an option to export the subject features, an image version of the polar representation of the subject features, and/or the polar image.

TABLE 2
An example of patient clinical profile original and color-coded values.

| Circle | Demographic/clinical | Original | |
|---|---|---|---|
| label | information | Values | Color codes |
| 1 | Age, yrs | 66 | Orange: 0.66 |
| 2 | Sex | Male | Blue: 0.3 |
| 3 | Body Mass Index, kg/m² | 30.12 | Orange: 0.69 |
| 4 | Smoking | Yes | White: 1 |
| 5 | Diabetes | No | Blue: 0.2 |
| 6 | Hypertension | No | Blue: 0.2 |
| 7 | Angina Pectoris | Yes | White: 1 |
| 8 | Ventricular Tachycardia | Yes | White: 1 |
| 9 | Prior Myocardial | No | Blue: 0.2 |
| | Infarction | | |
| 10 | Beta-blockers | Yes | White: 1 |
| 11 | ACE-inhibitors | No | Blue: 0.2 |
| 12 | Anti-arrhythmics | No | Blue: 0.2 |
| 13 | Diuretics | No | Blue: 0.2 |

FIG. 8 is an illustration of a third panel of the user interface, according to one example of the present disclosure. The third panel can present the predicted heart failure categories and their corresponding likelihoods. These categories include heart failure with preserved ejection fraction (HFpEF), heart failure with mid-range ejection fraction (HFmEF), and/or heart failure with reduced ejection fraction (HFrEF). In one example, the third panel may also present additional assessment of the heart failure prediction. In particular, the third panel present further analysis on the decision made by the machine learning model in an attention-based heat map. The attention-based heat map may include a polar representation using the same 1-hour segmentation of a 24-hour time period. The heat map may include regions of interest that may be derived from the decisions made by the machine learning model. Further, the third panel can present the HRV features and the subject features plotted over the 24-hour time period, allow the definition of a threshold in these plots to indicate the contributing HRV and subject features. The third panel can also include an option to export the prediction, the heatmap, and the plots.

As such, based on the third panel, the application provides additional assessment for heart failure patients. The application returns to the user the predicted heart failure category including HFpEF, HFmEF, and HFrEF. Furthermore, it provides further analysis on the decision made by the deep learning model in a form of the attention-based heatmap. The heatmap mimics the human perception in analyzing objects as it returns most important regions that derived the decisions made by the trained model. The application can further analyze these decisions and provide the user with complete line plots for per-hour importance and per-clinical features importance.

As illustrated with the above user interface, the embodiments allow the utilization of ECG/HRV data in a 2D manner instead of the conventional 1D representation. The embodiments allow for a better visual inspection of HRV variations in a selected proportion of time, thus, better evaluation of cardiac variations could be achieved. In addition, the embodiments integrate these variations with patient clinical information all in one 2D image instead of regular patient profiles stored in sheets as bulky data. The ability to create a color-coded clinical information simplifies the evaluation of patients with respect to their cardiac health condition, which would ensure better diagnosis when integrated altogether with the HRV variations all in one 2D polar image.

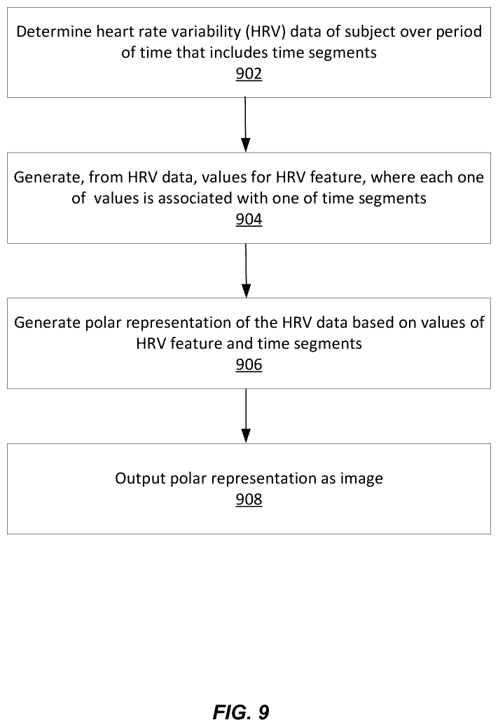

FIG. 9 illustrates an example of a flow for generating an HRV polar image, according to one example of the present disclosure. Operations of the flow can be performed by a computer system, such as the computer system 110. In the interest of clarity of explanation, the flow is described in connection with a single HRV feature. Nonetheless, operations of the flow can be repeated for multiple HRV features.

In an example, the flow includes operation 902, where the computer system determines HRV data of a subject over a period of time that includes the time segments. For instance, the computer system receives ECG data and extracts the HRV data therefrom or receives the HRV data directly. The time period can be predefined, such as to span 24 hours. The time segments can also be defined, such as each being 1-hour long.

In an example, the flow includes operation 904, where the computer system generates, from the HRV data, values of an HRV feature from, where each one of the values is associated with one of the time segments. For instance, the HRV feature may be selected via a user interface. The HRV data corresponding to each time segment is determined and a statistical measure is applied to generate a value for an HRV feature that corresponds to the time segment.

In an example, the flow includes operation 906, where the computer system generates a polar representation of the HRV data based on the values of the HRV feature and the time segments. For instance, the polar representation is a circle, where the time segments are evenly distributed around the circle and represented by lines radially extending from the center of the circle. The HRV feature value corresponding to a time segment is represented by a point on the corresponding line according to its value. The adjacent points are connected by edges.

In an example, the flow includes operation 908, where the computer system outputs the polar representation as an image. As described herein above, the polar representation can be converted into an edge image that is then used to generate a filled image, and the filled image is scaled resulting in an HRV polar image that the computer system outputs.

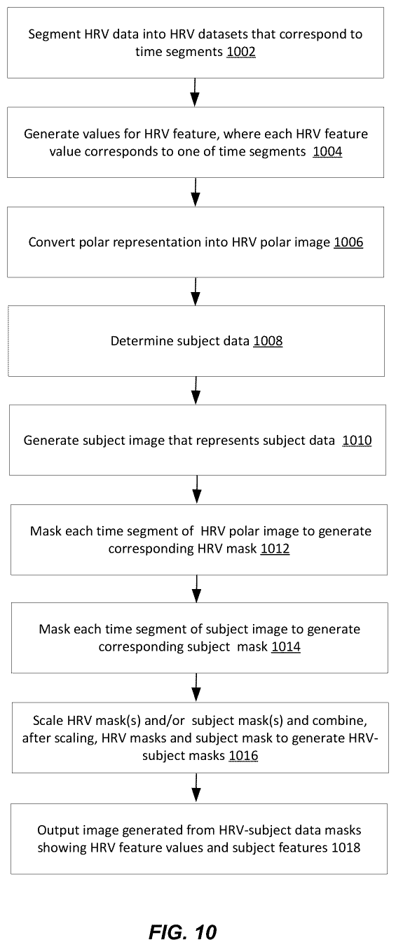

FIG. 10 illustrates an example of a flow for generating a polar representation of HRV data and subject data, according to one example of the present disclosure. Operations of the flow can be performed by a computer system, such as the computer system 110. Some or all of the operations can be used in conjunction with the flow of FIG. 9. Here also, in the interest of clarity of explanation, the flow is described in connection with a single HRV feature. Nonetheless, operations of the flow of FIG. 10 can be repeated for multiple HRV features.

In an example, the flow of FIG. 10 includes operation 1002, where the computer system segments HRV data into HRV datasets that correspond to time segments. The time segments can be predefined time segments. In one example, each time segment may be 1-hour long.

In one example, the flow includes operation 1004, wherein the computer system generates an HRV feature value that corresponds to a time segment based on the HRV dataset that corresponds to the time segment. The HRV feature can be a time domain feature (average normal-to-normal, average interval normal-to-normal, standard deviation of the normal-to-normal intervals, square root of the mean of the sum of squares of differences between adjacent normal-to-normal intervals, and/or percentage of normal-to-normal intervals more than 50 milliseconds). In one example, the HRV feature can be a frequency-domain feature (slope of the linear interpolation of the spectrum for frequencies less than the very-low frequency band upper bound, high frequency, normalized power, peak frequency of the high frequency band, power in the high frequency band, low frequency normalized power, peak frequency of the low frequency band, power in the low frequency band, ratio between the low frequency power and the high frequency power, total power in both frequency bands, very-low frequency normalized power, and/or power in the very-low frequency band). In another example, the HRV feature can include non-linear metrics (standard deviation of the normal-to-normal interval, de-trended fluctuation analysis for the low-scale slope, de-trended fluctuation analysis for the high-scale slope, the complexity of physiological time-series signals). In another example, The HRV features can include fragmentation metrics (percentage of inflection points in the normal-to-normal intervals, acceleration and deceleration segments inverse average length, percentage of short segments, and/or percentage of alternation segments).

In one example, the flow includes operation 1006, where the computer system converts a polar representation of the HRV feature values into an HRV polar image. For instance, the polar representation includes edges that connect the point that represent HRV feature values in the polar representation. An edge image can be generated to show the edges as a boundary that starts at the first time segment and ends back the first time segment. Remaining data from the polar representation need not be shown in the edge image. A filled image can be generated from the edge image by filling in the area defined by the boundary. The filled image can be a binary image, where one color is used for the area and another color is used for the remaining portion of the image. The filled image is scaled using a maximum allowable value, resulting in an HRV polar image for the HRV feature.

In one example, the flow includes operation 1008, where the computer system determines subject data. For example, demographic data and/or clinical data can be received via a user interface and/or imported via the user interface.

In one example, the flow includes operation 1010, where the computer system generates a subject image that represents the subject data. For instance, the subject data can be converted into subject features, where each feature is assigned a value given a coding rule. A polar representation can be generated based on the subject feature rules, where this representation uses rings that represent a full circle with the maximum possible subject feature value of one and that are segmented into the time segments. Color coding can be used to show the values of the subject features.

In one example, the flow includes operation 1012, where the computer system masks each time segment of the HRV polar image to generate a corresponding HRV mask. Each HRV mask can be a portion of the HRV polar image, where the portion corresponds to one of the time-segments (e.g., a time slice or pie of the HRV polar image).

In one example, the flow includes operation 1014, where the computer system masks each time segment on the subject image to generate a subject mask. Each subject mask can be a portion of the subject image, where the portion corresponds to one of the time-segments (e.g., a time slice or pie of the subject image).

In one example, the flow includes operation 1016, where the computer system scales the HRV mask(s) and/or the subject mask(s) and combines, after scaling, the HRV masks and the subject masks to generate the HRV-subject masks. The combination uses pairs of HRV mask-subject mask, where each pair corresponds to the same time segment. The combination could include a multiplication such that an HRV mask (e.g., with each pixel therein having a value of “1”) can be colored by the subject mask (e.g., where each pixel is updated to take the value of the corresponding pixel from the subject mask).

In one example, the flow include operation 1018, where the computer system outputs the image generated from the HRV-subject masks showing HRV feature values and the subject feature values. For instance, the HRV-subject masks can be assembled according to their corresponding time segments (e.g., an HRV-subject mask corresponding to the time segment of “hour 1” is placed in between an HRV-subject mask corresponding to the time segment of “hour 24” and an HRV-subject mask corresponding to the time segment of “hour 2”).

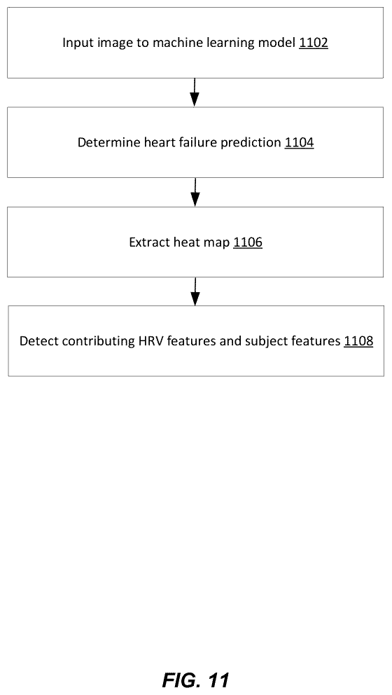

FIG. 11 illustrates an example of a flow for using a machine learning model to predict a heart failure, according to one example of the present disclosure. Operations of the flow can be performed by a computer system, such as the computer system 110. Some or all of the operations can be used in conjunction with the flows of FIGS. 9 and 10. Here also, in the interest of clarity of explanation, the flow is described in connection with a single image input. Nonetheless, the input can include multiple images, each corresponding to an HRC feature.

In one example, the flow includes operation 1102, where the computer system inputs an image to the machine learning model. The image can be the image generated from the HRV-subject masks described in connection with FIG. 10.

In one example, the flow includes operation 1104, where the computer system determines a heart failure prediction. The prediction can be included in an output of the machine learning model and can include a likelihood per heart failure category. These categories include HFpEF, HFmEF, and/or HFrEF.

In one example, the flow includes operation 1106, where the computer system extracts a heatmap. In one example, the extracted heatmap may be generated by determining values from a layer of the machine learning model (e.g., a max pooling layer). These values can be organized in a polar representation that uses the same time segments in order to represent the heatmap.

In one example, the flow includes operation 1108, where the computer system detects contributing HRV feature values and contributing subject features to the machine learning output. For instance, given the heatmap, particular time segments are determined. A threshold can be used for comparison to values of the HRV features and/or subject features during these time segments. Based on the comparison, a contributing factor can be determined (e.g., if a value of an HRV feature exceeds the threshold during a time segment, then that HRV feature is one of the contributing factors).

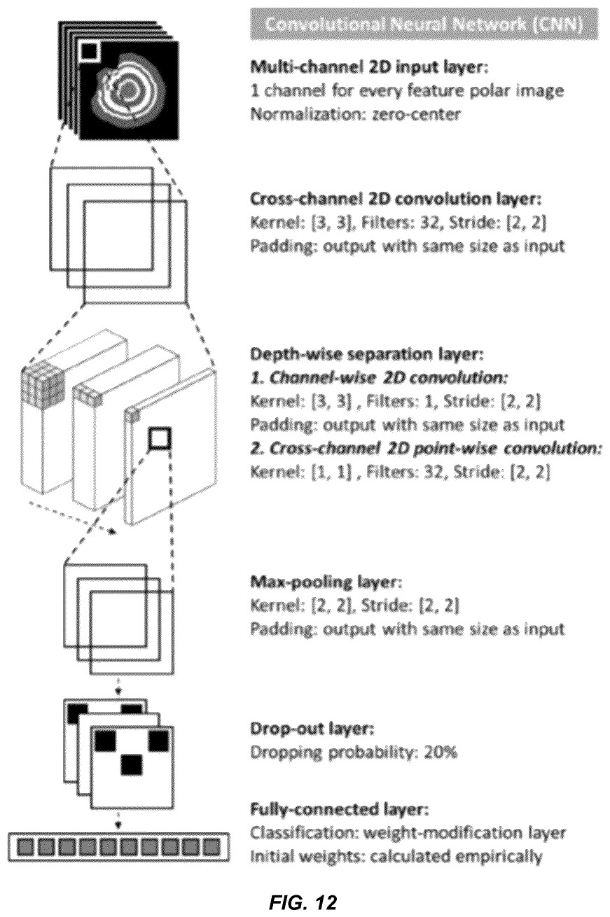

FIG. 12 illustrates an example of a machine learning model usable to generate heart failure predictions, according to one example of the present disclosure. The machine learning model has a structure (e.g., a convolutional neural network structure) that allows it to learn from HRV features altogether as well as individually. The structure includes a multi-channel two-dimensional (2D) input layer that passes the best selected images first to a cross-channel convolutional layer then to a channel-wise convolutional layer. To reduce complexity, the machine learning model uses a max-pooling layer followed by a drop-out layer (e.g., 20% probability) to prevent over-fitting. The last layer in the machine learning model (e.g., a fully-connected layer) includes a weight-modified layer with initial weights that were calculated empirically to handle slight dataset imbalances. The multi-channel 2D input layer can have one channel for every feature polar image and uses a zero-center normalization. The cross-channel 2D convolution layer uses a [3, 3] kernel, thirty-two filters and a [2, 2] stride. The padding allows an output having a same size as the input. A depth-wise separation layer uses a channel-wise 2D convolution (with a [3, 3] kernel, thirty-two filters and a [2, 2] stride) and a cross-channel 2D point-wise convolution (with a [1, 1] kernel, thirty-two filters and a [2, 2] stride). The max pooling layer has a [2, 2] kernel and a [2, 2] stride. The padding allows an output having a same size as the input. The drop out layer allows the 20% probability-based dropping. The fully connected layer allows a classification that uses weight-modification layer with initial weights calculated empirically.

As such, the machine learning model (e.g., a convolutional neural network) is implemented to start with a multi-channel 2D input layer that accepts a variable number of HRV features. Each feature is assigned to a channel and a zero-center normalization is applied accordingly. Then, the network applies an initial cross-channel 2D convolutions with kernel size of [3, 3], 32 filters, and stride of [2, 2] with padded output similar in size to the input. Another depth-wise convolution is applied to the outputs of the first convolution to ensure a channel-wise 2D feature extraction mechanism. The layer has a single filter with kernel size of [3, 3] and stride of [2, 2]. The padding is similar to the previous convolutional step. The depthwise convolution proceeds with cross-channel 2D point-wise convolution with kernel of [1, 1], 32 filters, and stride of [2, 2]. After applying convolutions, a max-pooling mechanism is applied to reduce the dimensionality and complexity in the network with a kernel size of [2, 2] and stride of [2, 2]. To prevent the network from over-fitting, a 20% drop-out layer is added. For classification, the fully-connected layer is used with soft-max and weight-modified layers that calculates class weights empirically.

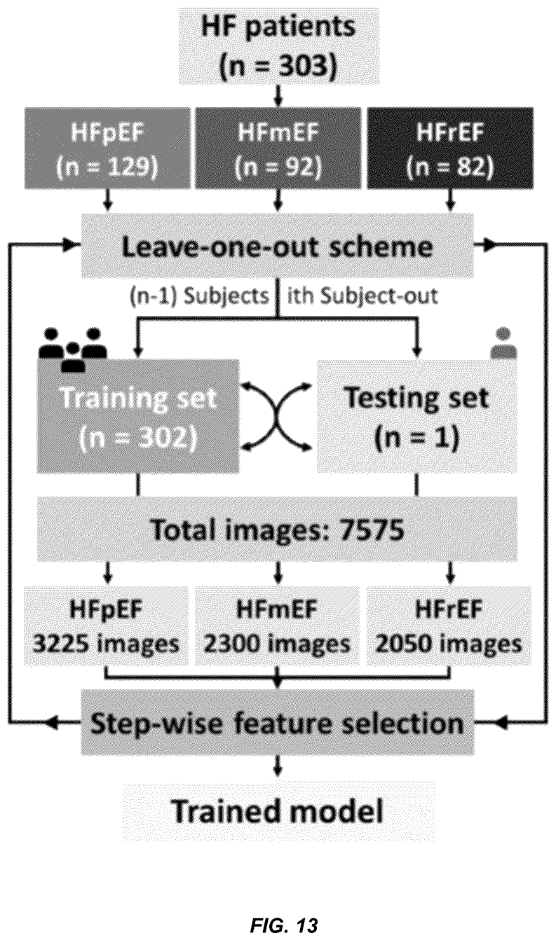

FIG. 13 illustrates an example of a training the machine learning model to generate heart failure predictions, according to one example of the present disclosure. The training includes a total of “n” patients (shown as 303) and follows a leave-one-out (LOO) cross-validation scheme, where on each iteration, an ith subject is used for testing and the remaining n−1 subjects are used for training. Instead of training the network on the whole set of HRV features (25 HRV features, for 303 patients, resulting in 7575 HRV images) all at once, features that were of high discriminative ability in exhibiting differences between the three LVEF-based heart failure groups are determined. To achieve this, a step-wise feature selection approach is followed. In this approach, twenty-five iterations are used based on the total number of HRV features. At the first iteration, each feature is used to train the model and output the corresponding classification accuracy. After picking the highest performing feature, it is added to the best-features set that is going to be used on all the following iterations. On the second iteration, the process continues by adding HRV features one by one (step-by-step) to the best-features set and observing the classification performance accordingly. This iterative process keeps running until all features are ranked based on their impact to the overall classification process. At the end, the iteration that gave the highest accuracy is selected and all features included in the best-features set up to this iteration are selected as optimal features for the training and classification process.

Such set of HRV and/or subject features can be used at the inference stage for a subject. In particular, an HRV polar image can be generated for each HRV feature in the set from HRV data of the subject. The generated HRV polar images can be input to an instance of the trained machine learning model to generate a heart failure prediction for the subject

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the disclosure to the specific form or forms disclosed, but on the contrary, the intention is to cover all modifications, alternative constructions, and equivalents falling within the spirit and scope of the disclosure, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Various embodiments of this disclosure are described herein, including the best mode known to the inventors for carrying out the disclosure. Variations of those embodiments may become apparent to those of ordinary skill in the art upon reading the foregoing description. The inventors expect skilled artisans to employ such variations as appropriate and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A computer-implemented method comprising:
determining heart rate variability (HRV) data of a subject over a period of time that includes time segments;
generating, from the HRV data, values for an HRV feature, wherein each one of the values is associated with one of the time segments;
generating, based on the values of the HRV feature and the time segments, a polar representation of the HRV data; and
outputting the polar representation as an image.

2. The computer-implemented method of claim 1, further comprising:
determining subject data of the subject, the subject data comprising at least one of demographic data or clinical data; and
representing the subject data in the polar representation, wherein the image shows the values of HRV feature and values that represent the subject data.

3. The computer-implemented method of claim 2, wherein edges of the polar representation correspond to HRV feature variations between the time segments, wherein the image includes lines that represent time segments, and wherein areas between the lines correspond to color-coded values that represent the subject data.

4. The computer-implemented method of claim 1 further comprising:
segmenting the HRV data into HRV datasets that correspond to the time segments, wherein each value of the HRV feature corresponds to a time segment and is generated from an HRV dataset that corresponds to the time segment.

5. The computer-implemented method of claim 4 further comprising:
converting the polar representation into an edge image;
converting the edge image into a filled image; and
converting the filled image into a scaled image based on a set of maximum possible values that the scaled image can show, wherein the image is outputted as the scaled image.

6. The computer-implemented method of claim 1, further comprising:
determining subject data of the subject, the subject data comprising at least one of: demographic data or clinical data;
generating values for the subject data based on a coding rule;
generating, based on the values generated for the subject data, a subject image that represents the subject data;
generating an HRV polar image based on the polar representation;
masking each time segment of the HRV image to generate a corresponding HRV mask;
masking each time segment of the subject image to generate a corresponding subject mask;
scaling one or more of the subject masks and/or one or more of the HRV masks; and
combining, after the scaling, the HRV masks and the subject masks to generate HRV-subject masks, wherein the image is generated based on the HRV-subject masks.

7. The computer-implemented method of claim 6, wherein the subject data comprises age, gender, body mass index, smoking, diabetes, hypertension, angina pectoris, ventricular tachycardia, prior myocardial infarction, beta-blockers, ACE-inhibitors, anti-arrhythmics, and diuretics.

8. The computer-implemented method of claim 1 further comprising:
generating an input to a machine learning model based on the image or the polar representation; and
determining a heart failure prediction based on an output of the machine learning model.

9. The computer-implemented method of claim 8, wherein the heart failure prediction comprises a plurality of heart failure types and a likelihood of each one of the plurality of heart failure types.

10. The computer-implemented method of claim 8, wherein the image further shows subject data, wherein the subject data comprises at least one of demographic data or clinical data, and wherein the input is generated based on the image and represents the values of HRV features and values that represent the subject data.

11. The computer-implemented method of claim 10 further comprising:
extracting a heatmap from a layer of the machine learning model; and
identifying, based on the heatmap, a contributing factor to the heart failure prediction, the contributing factor comprising at least one contributing HRV feature values or contributing subject features.

12. The computer-implemented method of claim 10 further comprising:
presenting the HRV data and the polar representation in a first panel of a user interface of an application.

13. The computer-implemented method of claim 12 further comprising:
receiving the subject data via a second panel of the user interface;
representing the subject data in the polar representation;
generating another image that represents the subject data; and
presenting the image and the other image in the second panel, the image showing the polar representation that includes the values of the HRV feature and the values that represent the subject data, and wherein the other image shows the values of the subject data.

14. The computer-implemented method of claim 13 further comprising:
presenting the heart failure prediction in a third panel of the user interface of the application.

15. The computer-implemented method of claim 14 further comprising:
presenting, in the third panel, contributing factors to the heart failure prediction.

16. A computer system comprising:
one or more processors; and
one or more memory storing computer-readable instructions that, upon execution by the one or more processors, configure the computer system to:
determine heart rate variability (HRV) data of a subject over a period of time that includes time segments;
generate, from the HRV data, values for an HRV feature, wherein each one of the values is associated with one of the time segments;
generate, based on the values of the HRV feature and the time segments, a polar representation of the HRV data; and
output the polar representation as an image.

17. The computer system of claim 16, wherein the execution of the computer-readable instructions further configures the computer system to:
determine subject data, the subject data comprising at least one of demographic data or clinical data; and
represent the subject data in the polar representation, wherein the image shows the values of the HRV feature and values that represent the subject data.

18. The computer system of claim 17, wherein the execution of the computer-readable instructions further configures the computer system to:
generate an input to a machine learning model based on at least one of the image or the polar representation; and
determine a heart failure prediction based on an output of the machine learning model.

19. One or more computer-readable storage media storing instructions that, upon execution on a computer system, cause the computer system to perform operations comprising:
determining heart rate variability (HRV) data of a subject over a period of time that includes time segments;
generating, from the HRV data, values of an HRV feature, wherein each one of the values is associated with one of the time segments;
generating, based on the values of the HRV feature and the time segments, a polar representation of the HRV data; and
outputting the polar representation as an image.

20. The one or more computer-readable storage media of claim 19, wherein the operations further comprise:
determining subject data of the subject, the subject data comprising at least one of demographic data or clinical data;
representing the subject data in the polar representation, wherein the image shows the values of the HRV feature and values that represent the subject data;
generating an input to a machine learning model based on at least one of the image or the polar representation; and
determining a heart failure prediction based on an output of the machine learning model.
